(19) United States
(12) Patent Application Publication
(10) Pub. No.: US 2019/0350882 A1
(43) Pub. Date: Nov. 21, 2019

(54) METHODS OF TREATING ATTENTION DEFICIT HYPERACTIVITY DISORDER

(71) Applicant: Ovid Therapeutics, Inc., New York, NY (US)

(72) Inventor: Matthew During, Weston, CT (US)

(21) Appl. No.: 16/415,075

(22) Filed: May 17, 2019

Related U.S. Application Data
(60) Provisional application No. 62/673,216, filed on May 18, 2018.

(51) Int. Cl. A61K 31/145; A61P 25/22

(52) U.S. Cl. CPC A61K 31/145; A61P 25/22

(57) ABSTRACT

Methods and compositions for treating Attention-deficit/hyperactivity disorder are provided which include administering to a patient in need thereof captodiamine or a pharmaceutically acceptable salt thereof.

### CROSS-REFERENCE TO RELATED APPLICATION

This application claims benefit and priority to U.S. Provisional Application No. 62/673,216, filed May 18, 2018, which is incorporated herein by reference in its entirety.

### TECHNICAL FIELD

Methods of using a composition including captodiamine, a derivative thereof, or a pharmaceutically acceptable salt thereof for the treatment of attention deficit hyperactivity disorder.

### BACKGROUND

Captodiamine (also known as captodiame) (2-[(4-butylsulfanylphenyl)-phenyl-methyl]sulfanyl-N,N-dimethylethanamine) was developed in the 1950's and is a derivative of the antihistamine diphenhydramine. Captodiamine is also known as 2-[(4-butylsulfanylphenyl)-phenylmethyl]sulfanyl-N,N-dimethylethanamine. The chemical formula for captodiamine is C₂₁H₂₉NS₂ and its CAS number is 486-17-9. Without limitation, the compound may also be known under the following synonyms: 2-[(p-(Butylthio)-alpha-phenylbenzyl)thio)-N,N-dimethylethylamine, 4-06-00-06672 (Beilstein Handbook Reference), 486-17-9, BRN 2625367, Captodiame, Captodiamin, Captodiamo [INN-Spanish], Captodiamum [INN-Latin], Captodramin, Captodramine, Covatin, Covatix, EINECS 207-629-1, Ethanamine, 24(4-(butylthio)phenyl)phenylmethyl)thio)-N,N-dimethyl-(9CI), Ethanamine, 2-[[[4-(butylthio)phenyl]phenylmethyl]thio]-N,N-dimethyl-, ethylamine, 2-[(p-(butylthio)-alpha-phenylbenzyl)thio)-n,n-dimethyl-, Kaptodiamin [Czech], N 68, p-Butylmercaptobenzhydryl-beta-dimethylamino-ethylsulphide, and VUFB2350.

Captodiamine was identified in a class of compounds designated as sedatives and antispasmodics. See, e.g., U.S. Pat. No. 2,830,083, incorporated herein by reference. It was subsequently developed for treatment of anxiety disorders and marketed as an anxiolytic. The drug is not a potent hypnotic and appeared to be safe and well tolerated. Although no formal double blind randomized studies were carried out on captodiamine when it was initially marketed, it was widely prescribed.

Captodiamine was marketed as SUVREN® by Ayerst at the time notwithstanding the fact that there was a paucity of published studies. In the early 1960's the FDA DESI (Drug Evaluation & Safety Initiative) program was implemented, i.e., drugs which did not have compelling evidence of efficacy, strong proponents, or external champions, were essentially struck off the register, or removed from the approved list. Captodiamine was one such drug, and essentially disappeared.

In 1999, a comparative study on the effects of captodiamine and lorazepam on car driving ability was conducted (Mercier-Guyon, et al., Clin Drug Invest, 17 (6): 451-459 (1999)). Captodiamine, as compared to lorazepam, reportedly improved the concentration and dexterity of individuals when driving, without inducing a tendency to drowsiness. In another study, captodiamine treatment was associated with lower severity of benzodiazepine withdrawal symptoms as compared to placebo (Mercier-Guyon et al., Curr Med Res Opin., 20 (9):1347-1355 (2004)).

In 2013, a group from University College Dublin, published a paper in which they reported that captodiamine had activity as a sigma-1 receptor agonist as well as a 5-HT_{2C} antagonist and dopamine D3 agonist (Ring and Regan, Journal of Psychopharmacology, 27 (10):930-939 (2013)). Captodiamine was shown to increase brain-derived neurotrophic factor (BDNF) protein levels in the hypothalamus, but not in the cortex or the hippocampus. U.S. Pat. No. 8,461,389 describes use of captodiamine in the treatment of anxiety and/or depression associated with an affective disorder and/or symptoms associated with cognitive impairment disorder.

Attention-deficit/hyperactivity disorder (ADHD), also known as attention deficit disorder (ADD), is a brain disorder marked by an ongoing pattern of inattention and/or hyperactivity-impulsivity that interferes with functioning or development. An estimated 15 million people in America have ADHD. Without identification and proper treatment, ADHD may have serious consequences, including school failure, family stress and disruption, depression, problems with relationships, substance abuse, delinquency, accidental injuries and job failure.

ADHD may be divided into three subtypes, according to the main features associated with the disorder: inattentiveness, impulsivity, and hyperactivity. Inattention may include, e.g., a person wanders off task, lacks persistence, has difficulty sustaining focus, and is disorganized; and these problems are not due to defiance or lack of comprehension. Hyperactivity may include, e.g., a person seems to move about constantly, including in situations in which it is not appropriate; or excessively fidgets, taps, or talks. In adults, it may be extreme restlessness or wearing others out with constant activity. Impulsivity may include, e.g., a person makes hasty actions that occur in the moment without first thinking about them and that may have high potential for harm; or a desire for immediate rewards or inability to delay gratification. An impulsive person may be socially intrusive and excessively interrupt others or make important decisions without considering the long-term consequences.

The three subtypes are: 1) predominantly combined type, predominantly inattentive type, and predominantly hyperactive-impulsive type. These subtypes take into account that some people with ADHD have little or no trouble sitting still or inhibiting behavior, but may be predominantly inattentive and, as a result, have great difficulty getting or staying focused on a task or activity. Others with ADHD may be able to pay attention to a task but lose focus because they may be predominantly hyperactive-impulsive and, thus, have trouble controlling impulse and activity. The most prevalent subtype is the combined type. These people will have significant symptoms of all three characteristics.

The most common type of medications used for treating ADHD are stimulants. However, there are risks and side effects associated with stimulants, especially when misused or taken in excess of the prescribed dose. For example, stimulants can raise blood pressure and heart rate and increase anxiety. Certain non-stimulants can be used to treat ADHD. For example, atomoxetine (Strattera™), is not a stimulant, but it has been linked to seizures and irregular heartbeats.

There remains a need for improved methods of treating ADHD.

### SUMMARY

Methods of treating Attention-deficit/hyperactivity disorder are provided which include administering to a patient in need thereof a pharmaceutical composition including captodiamine or a pharmaceutically acceptable salt thereof. In embodiments, methods of treating Attention-deficit/hyperactivity disorder include administering captodiamine or a pharmaceutically acceptable salt thereof to a patient in need thereof to provide improvement in the Attention-deficit/hyperactivity disorder in the patient. In embodiments, methods of treating Attention-deficit/hyperactivity disorder include administering captodiamine or a pharmaceutically acceptable salt thereof to a patient in need thereof to provide improvement in the Attention-deficit/hyperactivity disorder in the patient the next day after administration. In embodiments, administration of captodiamine or a pharmaceutically acceptable salt thereof provides reduction in one or more of hyperactivity, impulsiveness, inattentiveness, or a combination of symptoms in a patient diagnosed with Attention-deficit/hyperactivity disorder.

In embodiments, the patient is administered a pharmaceutical composition including 0.1 mg to 1500 mg of captodiamine or a pharmaceutically acceptable salt thereof. In embodiments, the patient is administered a pharmaceutical composition including 1 mg to 500 mg of captodiamine or a pharmaceutically acceptable salt thereof. In embodiments, the patient is administered a pharmaceutical composition including 50 mg to 250 mg of captodiamine or a pharmaceutically acceptable salt thereof. In embodiments, the patient is administered a pharmaceutical composition including 10 mg to 100 mg of captodiamine or a pharmaceutically acceptable salt thereof In embodiments, a pharmaceutical composition including from 1 mg to 1500 mg of captodiamine or a pharmaceutically acceptable salt thereof is administered within a 24-hour period. In embodiments, the total amount of captodiamine or a pharmaceutically acceptable salt thereof administered to the patient in a twenty-four hour period is between 1 mg and 1500 mg. In embodiments, the total amount of captodiamine or a pharmaceutically acceptable salt thereof administered to the patient in a twenty-four hour period is between 1 mg and 500 mg. In embodiments, a pharmaceutical composition including captodiamine or a pharmaceutically acceptable salt thereof is administered from one to four times a day.

In embodiments, administering a pharmaceutical composition including captodiamine or a pharmaceutically acceptable salt thereof is accomplished via one or more of the following routes: oral, buccal, sublingual, rectal, topical, intranasal, and parenteral.

In embodiments, administering a pharmaceutical composition including captodiamine or a pharmaceutically acceptable salt thereof to a patient in need thereof provides improvement in at least one of the following symptoms: inattentiveness, impulsivity, and hyperactivity.

### DETAILED DESCRIPTION

Provided herein are methods and compositions for treating attention-deficit/hyperactivity disorder (ADHD) by administering to a patient in need thereof a pharmaceutical composition including captodiamine or a pharmaceutically acceptable salt thereof. Provided herein are methods and compositions for treating attention-deficit/hyperactivity disorder (ADHD) by administering to a patient in need thereof captodiamine or a pharmaceutically acceptable salt thereof. In embodiments, the methods and compositions described herein include captodiamine or a pharmaceutically acceptable salt thereof.

In embodiments, the methods and compositions are used to increase attention, decrease impulsiveness and decrease hyperactivity in patients with ADHD. For example, the methods and compositions may be used to treat symptoms including moderate-to-severe distractibility, short attention span, hyperactivity, emotional lability, and/or impulsivity. In embodiments, the methods and compositions may be used for treating attention function and impulsiveness in ADHD. In embodiments, the methods and compositions may be used for improving attention function in ADHD. In embodiments, the methods and compositions may be used for decreasing impulsiveness in ADHD.

Symptoms of ADHD addressed by the methods and compositions herein may be characterized by numerous features including chronic history of short attention span, distractibility, emotional lability, impulsivity, and moderate-to-severe hyperactivity. Nonlocalizing (soft) neurological signs, learning disability, and abnormal EEG may or may not be present, and a diagnosis of central nervous system dysfunction may or may not be warranted. Thus, the methods and compositions may be used to increase the ability to pay attention, stay focused on an activity, and control behavior problems. The methods can also help the patient organize tasks and improve listening skills.

The methods and compositions described herein may be useful for treating children and infants. In embodiments, the patient of the disclosed method is a preschooler, a school-age child, a tween, or a teenager. In embodiments, the patient is 18 years old or younger, 12 years old or younger, 10 years old or younger, 8 years old or younger, 6 years old or younger, 4 years old or younger, 2 years old or younger, 1 year old or younger. In embodiments, the patient is an adult that is over eighteen years old.

Accordingly, a patient in need thereof is a patient who has been diagnosed with ADHD. A diagnosis of Attention Deficit Hyperactivity Disorder (ADHD; DSM-IV) implies the presence of hyperactive-impulsive or inattentive symptoms that cause impairment and may be present before age 7 years. The symptoms should cause clinically significant impairment, e.g., in social, academic, or occupational functioning, and be present in 2 or more settings, e.g., school (or work) and at home. The symptoms should not be better accounted for by another mental disorder. For the Inattentive Type, at least 6 of the following symptoms must have persisted for at least 6 months: lack of attention to details/careless mistakes; lack of sustained attention; poor listener; failure to follow through on tasks; poor organization; avoids tasks requiring sustained mental effort; loses things; easily distracted; forgetful. For the Hyperactive-Impulsive Type, at least 6 of the following symptoms must have persisted for at least 6 months: fidgeting/squirming; leaving seat; inappropriate running/climbing; difficulty with quiet activities; “on the go;” excessive talking; blurting answers; can't wait turn; intrusive. The Combined Types requires both inattentive and hyperactive-impulsive criteria to be met.

Accordingly, captodiamine or a pharmaceutically acceptable salt thereof is used to treat a subject having ADHD. In embodiments, methods and compositions for treating ADHD include administering to a patient in need thereof a pharmaceutical composition including an effective amount of a captodiamine or a pharmaceutically acceptable salt thereof. The patient may be an animal, e.g., mammal, e.g., human, etc. As used herein, the terms “treat”, “treatment” or “treating” encompass any manner in which the symptoms or pathology of a condition, disorder or disease associated with ADHD are ameliorated or otherwise beneficially altered. In embodiments, “treat”, “treatment” or “treating” can refer to inhibiting a disorder, disease or condition, e.g., arresting or reducing its development or at least one clinical or subclinical symptom thereof. In embodiments, “treat”, “treatment” or “treating” can refer to relieving the disorder, disease or condition, e.g., causing regression of the disorder, disease or condition or at least one of its clinical or subclinical symptoms. In embodiments, “treating ADHD” means ameliorating, beneficially altering and/or providing relief from one or more of the symptoms of ADHD. The benefit to a patient being treated may be statistically significant, mathematically significant, or at least perceptible to the subject and/or the physician.

In embodiments, the terms “effective amount” or “therapeutically effective amount” refer to an amount of a compound, material, composition, medicament, or other material that is effective to achieve a particular pharmacological and/or physiologic effect in connection with ADHD symptoms such as, but not limited to, one or more of the following:
A. reducing or eliminating symptoms of inattention such as:
   1. overlooking or missing details, making careless mistakes in schoolwork, at work, or during other activities
   2. having problems sustaining attention in tasks or play, including conversations, lectures, or lengthy reading
   3. not seeming to listen when spoken to directly
   4. not following through on instructions and failing to finish schoolwork, chores, or duties in the workplace or starting tasks but quickly losing focus and getting sidetracked easily
   5. having problems organizing tasks and activities, such as what to do in sequence, keeping materials and belongings in order, having messy work and poor time management, and failing to meet deadlines
   6. avoiding or disliking tasks that require sustained mental effort, such as schoolwork or homework, or for teens and older adults, preparing reports, completing forms or reviewing lengthy papers
   7. losing things necessary for tasks or activities, such as school supplies, pencils, books, tools, wallets, keys, paperwork, eyeglasses, and cell phones
   8. being easily distracted by unrelated thoughts or stimuli
   9. being forgetful in daily activities, such as chores, errands, returning calls, and keeping appointments
B. Reducing symptoms of hyperactivity and impulsivity such as:
   1. fidgeting and squirming
   2. leaving locations in situations when staying is expected, such as in the classroom or in the office
   3. running or dashing around or climbing in situations where it is inappropriate or, in teens and adults, often feeling restless
   4. being unable to play or engage in hobbies quietly
   5. being constantly in motion or “on the go,” or act as if “driven by a motor”
   6. talking nonstop
   7. blurting out an answer before a question has been completed, finishing other people's sentences, or speaking without waiting for a turn in conversation
   8. having trouble waiting his or her turn
   9. interrupting or intruding on others, for example in conversations, games, or activities
   10. making important decisions without considering long term consequences
   11. engaging in reckless behavior and being accident prone

The dosage amount of captodiamine or a pharmaceutically acceptable salt thereof can vary according to a variety of factors such as subject-dependent variables (e.g., age, immune system, health, etc.), the disease or disorder being treated, as well as the route of administration and the pharmacokinetics of the agent being administered. For example, the effect of a composition including captodiamine or a pharmaceutically acceptable salt thereof on a particular symptom, pharmacologic, or physiologic indicator of ADHD can be compared to an untreated patient, or the condition of the subject prior to treatment. In embodiments, the symptom, pharmacologic, and/or physiologic indicator is measured in a patient prior to treatment, and again one or more times after treatment is initiated. In embodiments, the control is a reference level, or average determined based on measuring the symptom, pharmacologic, or physiologic indicator in one or more patients that do not have the disease or condition to be treated (e.g., healthy subjects). In embodiments, the effect of the treatment is compared to a conventional treatment that is known the art.

Effective treatment of ADHD herein may be established by showing reduction in the frequency or severity of one or more symptoms after a period of time compared with baseline. For example, after a baseline period of 1 month, the patients, after DSM-IV diagnosis of ADHD may be randomly allocated captodiamine or a pharmaceutical salt thereof, or placebo. The patient's schoolteacher may complete the Conners ADHD/DSM-IV Scale for Teachers (CADS-T) at baseline and the end of each week. The CADS-T assesses symptoms of hyperactivity and inattention. The change from baseline of the (CADS-T) scores during the last week of treatment may be analyzed as the primary efficacy parameter. Symptoms of ADHD may also be evaluated by community schoolteachers using the Inattention/Overactivity with Aggression (IOWA) Conners scale or using the SKAMP laboratory school rating scale, which specifically measures the classroom manifestation of ADHD. Patients treated with captodiamine or a pharmaceutical salt thereof may show a statistically significant improvement in symptom scores from baseline over patients who received placebo. The effectiveness of captodiamine or a pharmaceutical salt thereof for the treatment of ADHD may be established in other controlled studies.

Provided herein are dosing regimens that allow effective treatment of ADHD with potentially limited or substantially few negative side effects, e.g., convulsions and/or sleep disruption that may be associated with stimulant therapy. For example, methods are provided herein of treating ADHD in a patient in need thereof that may not cause sleep disruption. In embodiments, methods described herein may provide effective treatment of ADHD without interrupting Slow Wave Sleep. In embodiments methods of treating ADHD without causing insomnia or trouble falling asleep are provided.

In embodiments, methods include treating ADHD by administering to a patient in need thereof a pharmaceutical composition including about 0.01 mg to about 1000 mg of captodiamine or a pharmaceutically acceptable salt thereof. In embodiments, doses may be, e.g., in the range of about 0.1 to 1500 mg, 0.1 to 1250 mg, 0.1 to 1000 mg, 0.1 to 750 mg, 0.1 to 500 mg, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 1 mg, 1 to 1500 mg, 1 to 1000 mg, 1 to 500 mg, 1 to 300 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 5 to 1500 mg, 5 to 1000 mg, 5 to 500 mg, 5 to 300 mg, 5 to 250 mg, 5 to 200 mg, 5 to 175 mg, 5 to 150 mg, 5 to 125 mg, 5 to 100 mg, 5 to 75 mg, 5 to 50 mg, 5 to 30 mg, 5 to 25 mg, 5 to 20 mg, 5 to 15 mg, 5 to 10 mg, 10 to 1500 mg, 10 to 1000 mg, 10 to 500 mg, 10 to 300 mg, 10 to 250 mg, 10 to 200 mg, 10 to 175 mg, 10 to 150 mg, 10 to 125 mg, 10 to 100 mg, 10 to 75 mg, 10 to 50 mg, 10 to 30 mg, 10 to 25 mg, 10 to 20 mg, 10 to 15 mg, 15 to 1500 mg, 15 to 1000 mg, 15 to 500 mg, 15 to 300 mg, 15 to 250 mg, 15 to 200 mg, 15 to 175 mg, 15 to 150 mg, 15 to 125 mg, 15 to 100 mg, 15 to 75 mg, 15 to 50 mg, 15 to 30 mg, 15 to 25 mg, 15 to 20 mg, 20 to 1500 mg, 20 to 1000 mg, 20 to 500 mg, 20 to 300 mg, 20 to 250 mg, 20 to 200 mg, 20 to 175 mg, 20 to 150 mg, 20 to 125 mg, 20 to 100 mg, 20 to 75 mg, 20 to 50 mg, 20 to 30 mg, 20 to 25 mg, 25 to 1500 mg, 25 to 1000 mg, 25 to 500 mg, 25 to 300 mg, 25 to 250 mg, 25 to 200 mg, 25 to 175 mg, 25 to 150 mg, 25 to 125 mg, 25 to 100 mg, 25 to 75 mg, 25 to 50 mg, 25 to 30 mg, 30 to 1500 mg, 30 to 1000 mg, 30 to 500 mg, 30 to 300 mg, 30 to 250 mg, 30 to 200 mg, 30 to 175 mg, 30 to 150 mg, 30 to 125 mg, 30 to 100 mg, 30 to 75 mg, 30 to 50 mg, 35 to 1500 mg, 35 to 1000 mg, 35 to 500 mg, 35 to 300 mg, 35 to 250 mg, 35 to 200 mg, 35 to 175 mg, 35 to 150 mg, 35 to 125 mg, 35 to 100 mg, 35 to 75 mg, 35 to 50 mg, 40 to 1500 mg, 40 to 1000 mg, 40 to 500 mg, 40 to 300 mg, 40 to 250 mg, 40 to 200 mg, 40 to 175 mg, 40 to 150 mg, 40 to 125 mg, 40 to 100 mg, 40 to 75 mg, 40 to 50 mg, 50 to 1500 mg, 50 to 1000 mg, 50 to 500 mg, 50 to 300 mg, 50 to 250 mg, 50 to 200 mg, 50 to 175 mg, 50 to 150 mg, 50 to 125 mg, 50 to 100 mg, 50 to 75 mg, 75 to 1500 mg, 75 to 1000 mg, 75 to 500 mg, 75 to 300 mg, 75 to 250 mg, 75 to 200 mg, 75 to 175 mg, 75 to 150 mg, 75 to 125 mg, 75 to 100 mg, 100 to 1500 mg, 100 to 1000 mg, 100 to 500 mg, 100 to 300 mg, 100 to 250 mg, 100 to 200 mg, 100 to 175 mg, 100 to 150 mg, 100 to 125 mg, 125 to 1500 mg, 125 to 1000 mg, 125 to 500 mg, 125 to 300 mg, 125 to 250 mg, 125 to 200 mg, 125 to 175 mg, 125 to 150 mg, 150 to 1500 mg, 150 to 1000 mg, 150 to 500 mg, 150 to 300 mg, 150 to 250 mg, 150 to 200 mg, 150 to 175 mg, 175 to 1500 mg, 175 to 1000 mg, 175 to 500 mg, 175 to 300 mg, 175 to 250 mg, 175 to 200 mg, 200 to 1500 mg, 200 to 1000 mg, 200 to 500 mg, 200 to 300 mg, 200 to 250 mg, 250 to 1500 mg, 250 to 1000 mg, 250 to 500 mg, 250 to 300 mg, 7.5 to 15 mg, 2.5 to 5 mg, 1 to 5 mg, with doses of, e.g., about 0.25 mg, 0.5 mg, 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 10 mg, 15 mg, 20 mg, 25 mg, 30, mg, 35 mg, 40 mg, 45 mg, 50 mg, 75 mg, 100 mg, 125 mg, 150 mg, 175 mg, 200 mg, 225 mg, 250 mg, 275 mg, 300 mg, 400 mg and 500 mg being examples.

In embodiments, pharmaceutical compositions may include captodiamine or a pharmaceutically acceptable salt thereof in an amount of, e.g., about 0.01 to 500 mg, 0.1 to 500 mg, 0.1 to 450 mg, 0.1 to 300 mg, 0.1 to 250 mg, 0.1 to 200 mg, 0.1 to 175 mg, 0.1 to 150 mg, 0.1 to 125 mg, 0.1 to 100 mg, 0.1 to 75 mg, 0.1 to 50 mg, 0.1 to 30 mg, 0.1 to 25 mg, 0.1 to 20 mg, 0.1 to 15 mg, 0.1 to 10 mg, 0.1 to 5 mg, 0.1 to 1 mg, 0.5 to 500 mg, 0.5 to 450 mg, 0.5 to 300 mg, 0.5 to 250 mg, 0.5 to 200 mg, 0.5 to 175 mg, 0.5 to 150 mg, 0.5 to 125 mg, 0.5 to 100 mg, 0.5 to 75 mg, 0.5 to 50 mg, 0.5 to 30 mg, 0.5 to 25 mg, 0.5 to 20 mg, 0.5 to 15 mg, 0.5 to 10 mg, 0.5 to 5 mg, 0.5 to 1 mg, 1 to 500 mg, 1 to 450 mg, 1 to 300 mg, 1 to 250 mg, 1 to 200 mg, 1 to 175 mg, 1 to 150 mg, 1 to 125 mg, 1 to 100 mg, 1 to 75 mg, 1 to 50 mg, 1 to 30 mg, 1 to 25 mg, 1 to 20 mg, 1 to 15 mg, 1 to 10 mg, 1 to 5 mg, 5 to 500 mg, 5 to 450 mg, 5 to 300 mg, 5 to 250 mg, 5 to 200 mg, 5 to 175 mg, 5 to 150 mg, 5 to 125 mg, 5 to 100 mg, 5 to 75 mg, 5 to 50 mg, 5 to 30 mg, 5 to 25 mg, 5 to 20 mg, 5 to 15 mg, 5 to 10 mg, 10 to 500 mg, 10 to 450 mg, 10 to 300 mg, 10 to 250 mg, 10 to 200 mg, 10 to 175 mg, 10 to 150 mg, 10 to 125 mg, 10 to 100 mg, 10 to 75 mg, 10 to 50 mg, 10 to 30 mg, 10 to 25 mg, 10 to 20 mg, 10 to 15 mg, 15 to 500 mg, 15 to 450 mg, 15 to 300 mg, 15 to 250 mg, 15 to 200 mg, 15 to 175 mg, 15 to 150 mg, 15 to 125 mg, 15 to 100 mg, 15 to 75 mg, 15 to 50 mg, 15 to 30 mg, 15 to 25 mg, 15 to 20 mg, 20 to 500 mg, 20 to 450 mg, 20 to 300 mg, 20 to 250 mg, 20 to 200 mg, 20 to 175 mg, 20 to 150 mg, 20 to 125 mg, 20 to 100 mg, 20 to 75 mg, 20 to 50 mg, 20 to 30 mg, 20 to 25 mg, 25 to 500 mg, 25 to 450 mg, 25 to 300 mg, 25 to 250 mg, 25 to 200 mg, 25 to 175 mg, 25 to 150 mg, 25 to 125 mg, 25 to 100 mg, 25 to 75 mg, 25 to 50 mg, 25 to 30 mg, 30 to 500 mg, 30 to 450 mg, 30 to 300 mg, 30 to 250 mg, 30 to 200 mg, 30 to 175 mg, 30 to 150 mg, 30 to 125 mg, 30 to 100 mg, 30 to 75 mg, 30 to 50 mg, 40 to 500 mg, 40 to 450 mg, 40 to 400 mg, 40 to 250 mg, 40 to 200 mg, 40 to 175 mg, 40 to 150 mg, 40 to 125 mg, 40 to 100 mg, 40 to 75 mg, 40 to 50 mg, 50 to 500 mg, 50 to 450 mg, 50 to 300 mg, 50 to 250 mg, 50 to 200 mg, 50 to 175 mg, 50 to 150 mg, 50 to 125 mg, 50 to 100 mg, 50 to 75 mg, 75 to 500 mg, 75 to 450 mg, 75 to 300 mg, 75 to 250 mg, 75 to 200 mg, 75 to 175 mg, 75 to 150 mg, 75 to 125 mg, 75 to 100 mg, 100 to 500 mg, 100 to 450 mg, 100 to 300 mg, 100 to 250 mg, 100 to 200 mg, 100 to 175 mg, 100 to 150 mg, 100 to 125 mg, 125 to 500 mg, 125 to 450 mg, 125 to 300 mg, 125 to 250 mg, 125 to 200 mg, 125 to 175 mg, 125 to 150 mg, 150 to 500 mg, 150 to 450 mg, 150 to 300 mg, 150 to 250 mg, 150 to 200 mg, 200 to 500 mg, 200 to 450 mg, 200 to 300 mg, 200 to 250 mg, 250 to 500 mg, 250 to 450 mg, 250 to 300 mg, 300 to 500 mg, 300 to 450 mg, 300 to 400 mg, 300 to 350 mg, 350 to 500 mg, 350 to 450 mg, 350 to 400 mg, 400 to 500 mg, 400 to 450 mg, with 0.1 mg, 0.25 mg, 0.5 mg, 0.75 mg, 1 mg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg 175 mg, 200 mg, 225 mg, 250 mg, 275 mg, 300 mg, 325 mg, 350 mg, 375 mg, 400 mg, 425 mg, 450 mg, 475 mg, and 500 mg being examples.

In embodiments, dosages may be administered to a subject once, twice, three or four times daily, every other day, once weekly, or once a month. In embodiments, captodiamine or a pharmaceutically acceptable salt thereof is administered to a subject three times a day (e.g., at breakfast, lunch, and dinner), at a dose of 50 mg/administration (e.g., 150 mg/day). In embodiments, captodiamine or a pharmaceutically acceptable salt thereof is administered to a subject 250 mg/per day in one or more doses

In embodiments, the dosage of captodiamine or a pharmaceutically acceptable salt thereof is 0.01-100 mg/kg, 0.5-50 mg/kg, 0.5-10 mg/kg or 25-50 mg/kg once, twice, three times or four times daily. For example, in embodiments, the dosage is 0.5 mg/kg, 1 mg/kg, 5 mg/kg, 7.5 mg/kg, or 10 mg/kg once, twice, three times or four times daily. In embodiments, a patient is administered a total daily dose of 0.01 mg to 1500 mg of captodiamine or a pharmaceutically acceptable salt thereof once, twice, three times, four times daily. In embodiments, the total amount administered to a patient in 24-hour period is, e.g., 1 mg, 5 mg, 10 mg, 25 mg, 50 mg, 75 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, or 500 mg. In embodiments, the subject may be started at a low dose and the dosage is escalated.

Suitable dosage forms for captodiamine or a pharmaceutically acceptable salt thereof include, but are not limited to oral forms, such as tablets, hard or soft gelatin capsules, powders, granules and oral solutions, syrups or suspensions, troches, as well as the sublingual, buccal, intratracheal, intraocular, intranasal forms, forms adapted to inhalation, topical, transdermal, rectal forms such as suppositories, and implants for release of medication, parenteral forms, for example, intravenous, intra-arterial, intraperitoneal, intrathecal, intraventricular, intraurethrally, intrasternal, intracranial, intramuscularly or subcutaneously. In embodiments, for such parenteral administration, it may be in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

Pharmaceutical compositions herein are intended to include “dosage forms”. Dosage forms are intended to include “unit doses” and “dosages”. Pharmaceutical compositions herein may be provided with conventional release or modified release profiles. Modified release profiles include immediate release, delayed release, sustained release, and extended release profiles. In embodiments, a delayed release dosage form is one that releases a drug (or drugs) at a time other than promptly after administration. In embodiments, an extended release dosage form is one that allows at least a twofold reduction in dosing frequency as compared to that drug presented as a conventional dosage form (e.g. as a solution or prompt drug-releasing, conventional solid dosage form). In embodiments, a modified release dosage form is one for which the drug release characteristics of time course and/or location are chosen to accomplish therapeutic or convenience objectives not offered by conventional dosage forms such as tablets, capsules, solutions, suspensions, ointments, and suppositories, etc.

In embodiments, pharmaceutical compositions with different drug release profiles may be combined to create a two-phase or three-phase release profile. For example, pharmaceutical compositions may be provided with an immediate release and an extended release profile. In embodiments, pharmaceutical compositions may be provided with an extended release and delayed release profile. Such composition may be provided as pulsatile formulations, multilayer tablets, or capsules containing tablets, beads, granules, etc. Compositions may be prepared using a pharmaceutically acceptable “carrier” composed of materials that are considered safe and effective. The “carrier” includes all components present in the pharmaceutical formulation other than the active ingredient or ingredients. The term “carrier” includes, but is not limited to, diluents, binders, lubricants, glidants, disintegrants, fillers, and coating compositions.

In embodiments, methods of treating ADHD are provided which include administering to a subject in need thereof a pharmaceutical composition including captodiamine or a pharmaceutically acceptable salt thereof wherein the composition provides improvement in one or more symptoms of the ADHD for more than 6 hours after administration to the subject. In embodiments, methods of treating ADHD are provided which include administering to a subject in need thereof a pharmaceutical composition including captodiamine or a pharmaceutically acceptable salt thereof wherein the composition provides improvement in one or more symptoms of the ADHD for more than 8, 10, 12, 14, 16, 18, 20, 22 or 24 hours after administration to the subject. In embodiments, the pharmaceutical compositions provide improvement in one or more symptoms of the ADHD the next day after administration to the subject. For example, the pharmaceutical compositions may provide improvement in one or more symptoms of the ADHD for more than about, e.g., 2 hours, 4 hours, 6 hours, 8 hours, 10 hours, 12 hours, 14 hours, 16 hours, 18 hours, 20 hours, 22 hours or 24 hours after administration at bedtime or earlier, and waking from a night of sleep.

In embodiments, provided herein are methods of treating ADHD wherein the amount of captodiamine or a pharmaceutically acceptable salt thereof within the subject about 4 hours after administration of the pharmaceutical composition is between about 65% to about 85% of the administered dose. In embodiments, the amount of captodiamine or a pharmaceutically acceptable salt thereof within the subject after about, e.g., 6 hours, 8 hours, 10 hours, 12 hours, 15 hours, or 20 hours after administration of the pharmaceutical composition is between about 65% to about 85% of the administered dose.

In embodiments, the pharmaceutical compositions described herein may be administered once daily, twice daily, three times daily, four times daily, or every other day. In embodiments, the pharmaceutical compositions described herein may be administered by continuous infusion. In embodiments, a pharmaceutical composition described herein is provided to the subject in the morning. In embodiments, a pharmaceutical composition described herein is provided to the subject in the evening. In embodiments, a pharmaceutical composition described herein is provided to the subject once in the evening and once in the morning. In embodiments, a pharmaceutical composition described herein is provided to the subject once in the morning, once in the afternoon and once in the evening. In embodiments, the patient may be started at a low dose and the dosage is escalated. In this manner, it can be determined if the drug is well tolerated in the subject. Dosages can be lower for children than for adults.

In embodiments, as mentioned previously, pharmaceutical compositions herein may be provided with conventional release or modified release profiles.

In embodiments, pharmaceutical compositions herein are modified release dosage forms which provide modified release profiles. Modified release profiles may exhibit immediate release, delayed release, or extended release profiles. Extended release and sustained release may be used interchangeably herein. Conventional (or unmodified) release oral dosage forms such as tablets, capsules, suppositories, syrups, solutions and suspensions typically release medications into the mouth, stomach or intestines as the tablet, capsule shell or suppository dissolves, or, in the case of syrups, solutions and suspensions, when they are swallowed. The pattern of drug release from modified release (MR) dosage forms is deliberately changed from that of a conventional dosage form to achieve a desired therapeutic objective and/or better patient compliance. Types of MR drug products include orally disintegrating dosage forms (ODDFs) which provide immediate release, extended release dosage forms, delayed release dosage forms (e.g., enteric coated), and pulsatile release dosage forms.

An ODDF is a solid dosage form containing a medicinal substance or active ingredient which disintegrates rapidly, usually within a matter of seconds when placed upon the tongue. The disintegration time for ODDFs generally range from one or two seconds to about a minute. ODDFs are designed to disintegrate or dissolve rapidly on contact with saliva. This mode of administration can be beneficial to people who may have problems swallowing tablets whether it be from physical infirmity or psychiatric in nature. Some subjects with an eye disorder may exhibit such behavior. ODDF's can provide rapid delivery of medication to the blood stream through mucosa resulting in a rapid onset of action. Examples of ODDFs include orally disintegrating tablets, capsules and rapidly dissolving films and wafers.

Extended release dosage forms (ERDFs) have extended release profiles and are those that allow a reduction in dosing frequency as compared to that presented by a conventional dosage form, e.g., a solution or unmodified release dosage form. ERDFs provide a sustained duration of action of a drug. Suitable formulations which provide extended release profiles are well-known in the art. For example, coated slow release beads or granules (“beads” and “granules” are used interchangeably herein) in which captodiamine or a pharmaceutically acceptable salt thereof is applied to beads, e.g., confectioners nonpareil beads, and then coated with conventional release retarding materials such as waxes, enteric coatings and the like. In embodiments, beads can be formed in which captodiamine or a pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. Beads having different rates of release may be combined into a single dosage form to provide variable or continuous release. The beads can be contained in capsules or compressed into tablets.

In embodiments, modified dosage forms herein incorporate delayed release dosage forms having delayed release profiles. Delayed release dosage forms can include delayed release tablets or delayed release capsules. A delayed release tablet is a solid dosage form which releases a drug (or drugs) such as captodiamine or a pharmaceutically acceptable salt thereof at a time other than promptly after administration. A delayed release capsule is a solid dosage form in which the drug is enclosed within either a hard or soft soluble container made from a suitable form of gelatin, and which releases a drug (or drugs) at a time other than promptly after administration. For example, enteric-coated tablets, capsules, particles and beads are well-known examples of delayed release dosage forms. Enteric coated tablets, capsules and particles and beads pass through the stomach and release the drug in the intestine. In embodiments, a delayed release tablet is a solid dosage form containing a conglomerate of medicinal particles that releases a drug (or drugs) at a time other than promptly after administration. In embodiments, the conglomerate of medicinal particles are covered with a coating which delays release of the drug. In embodiments, a delayed release capsule is a solid dosage form containing a conglomerate of medicinal particles that releases a drug (or drugs) at a time other than promptly after administration. In embodiments, the conglomerate of medicinal particles are covered with a coating which delays release of the drug.

Delayed release dosage forms are known to those skilled in the art. For example, coated delayed release beads or granules in which captodiamine or a pharmaceutically acceptable salt thereof is applied to beads, e.g., confectioners nonpareil beads, and then coated with conventional release delaying materials such as waxes, enteric coatings and the like. In embodiments, beads can be formed in which captodiamine or a pharmaceutically acceptable salt thereof is mixed with a material to provide a mass from which the drug leaches out. In embodiments, the beads may be engineered to provide different rates of release by varying characteristics of the coating or mass, e.g., thickness, porosity, using different materials, etc. In embodiments, enteric coated granules of captodiamine or a pharmaceutically acceptable salt thereof can be contained in an enterically coated capsule or tablet which releases the granules in the small intestine. In embodiments, the granules have a coating which remains intact until the coated granules reach at least the ileum and thereafter provide a delayed release of the drug in the colon. Suitable enteric coating materials are well known in the art, e.g., Eudragit® coatings such methacrylic acid and methyl methacrylate polymers and others. The granules can be contained in capsules or compressed into tablets.

In embodiments, captodiamine or a pharmaceutically acceptable salt thereof is incorporated into porous inert carriers that provide delayed release profiles. In embodiments, the porous inert carriers incorporate channels or passages from which the drug diffuses into surrounding fluids. In embodiments, captodiamine or a pharmaceutically acceptable salt thereof is incorporated into an ion-exchange resin to provide a delayed release profile. Delayed action may result from a predetermined rate of release of the drug from the resin when the drug-resin complex contacts gastrointestinal fluids and the ionic constituents dissolved therein. In embodiments, membranes are utilized to control rate of release from drug containing reservoirs. In embodiments, liquid preparations may also be utilized to provide a delayed release profile. For example, a liquid preparation consisting of solid particles dispersed throughout a liquid phase in which the particles are not soluble. The suspension is formulated to allow at least a reduction in dosing frequency as compared to that drug presented as a conventional dosage form (e.g., as a solution or a prompt drug-releasing, conventional solid dosage form). For example, a suspension of ion-exchange resin constituents or microbeads.

In embodiments, pharmaceutical compositions described herein are suitable for parenteral administration, including, e.g., intramuscular (i.m.), intravenous (i.v.), subcutaneous (s.c.), intraperitoneal (i.p.), or intrathecal (i.t.). Parenteral compositions must be sterile for administration by injection, infusion, instillation or implantation into the body and may be packaged in either single-dose or multi-dose containers. In embodiments, liquid pharmaceutical compositions for parenteral administration to a subject include captodiamine or a pharmaceutically acceptable salt thereof in any of the respective amounts described above. In embodiments, the pharmaceutical compositions for parenteral administration are formulated as a total volume of about, e.g., 1 ml, 2 ml, 3 ml, 4 ml, 5 ml, 7.5 ml, 10 ml, 20 ml, 25 ml, 50 ml, 100 ml, 200 ml, 250 ml, or 500 ml. In embodiments, the compositions are contained in a bag, a glass vial, a plastic vial, or a bottle.

In embodiments, pharmaceutical compositions for parenteral administration include respective amounts described above captodiamine or a pharmaceutically acceptable salt thereof. In embodiments, pharmaceutical compositions for parenteral administration include about 0.05 mg to about 1500 mg captodiamine or a pharmaceutically acceptable salt thereof. In embodiments, pharmaceutical compositions for parenteral administration to a subject include an active substance, e.g., captodiamine or a pharmaceutically acceptable salt thereof, at a concentration of about 0.005 mg/ml to about 500 mg/ml. In embodiments, the pharmaceutical composition for parenteral administration includes captodiamine or a pharmaceutically acceptable salt thereof at a concentration of, e.g., about 0.05 mg/ml to about 50 mg/ml, about 0.1 mg/ml to about 50 mg/ml, about 0.1 mg/ml to about 10 mg/ml, about 0.05 mg/ml to about 25 mg/ml, about 0.05 mg/ml to about 10 mg/ml, about 0.05 mg/ml to about 5 mg/ml, or about 0.05 mg/ml to about 1 mg/ml. In embodiments, the pharmaceutical composition for parenteral administration includes captodiamine or a pharmaceutically acceptable salt thereof at a respective concentration of, e.g., about 0.05 mg/ml to about 15 mg/ml, about 0.5 mg/ml to about 10 mg/ml, about 0.25 mg/ml to about 5 mg/ml, about 0.5 mg/ml to about 7 mg/ml, about 1 mg/ml to about 10 mg/ml, about 5 mg/ml to about 10 mg/ml, or about 5 mg/ml to about 15 mg/ml.

In embodiments, a pharmaceutical composition for parenteral administration is provided wherein the pharmaceutical composition is stable for at least six months. In embodiments, the pharmaceutical compositions for ophthalmic or parenteral administration exhibit no more than about 5% decrease in active substance, e.g., captodiamine or a pharmaceutically acceptable salt thereof, e.g., in 3 months or 6 months. In embodiments, the amount of captodiamine or a pharmaceutically acceptable salt thereof degrades at no more than about, e.g., 2.5%, 1%, 0.5% or 0.1%. In embodiments, the degradation is less than about, e.g., 5%, 2.5%, 1%, 0.5%, 0.25%, 0.1%, for at least six months.

In embodiments, pharmaceutical compositions for parenteral administration are provided wherein the pharmaceutical composition remains soluble. In embodiments, pharmaceutical compositions for parenteral administration are provided that are stable, soluble, local site compatible and/or ready-to-use. In embodiments, the pharmaceutical compositions herein are ready-to-use for direct administration to a subject in need thereof.

The pharmaceutical compositions for parenteral administration provided herein may include one or more excipients, e.g., solvents, solubility enhancers, suspending agents, buffering agents, isotonicity agents, stabilizers or antimicrobial preservatives. When used, the excipients of the parenteral compositions will not adversely affect the stability, bioavailability, safety, and/or efficacy of captodiamine or a pharmaceutically acceptable salt thereof used in the composition. Thus, parenteral compositions are provided wherein there is no incompatibility between any of the components of the dosage form.

In embodiments, parenteral compositions including captodiamine or a pharmaceutically acceptable salt thereof include a stabilizing amount of at least one excipient. For example, excipients may be selected from the group consisting of buffering agents, solubilizing agents, tonicity agents, antioxidants, chelating agents, antimicrobial agents, and preservatives. One skilled in the art will appreciate that an excipient may have more than one function and be classified in one or more defined group.

In embodiments, parenteral compositions including captodiamine or a pharmaceutically acceptable salt thereof and an excipient wherein the excipient is present at a weight percent (w/v) of less than about, e.g., 10%, 5%, 2.5%, 1%, or 0.5%. In embodiments, the excipient is present at a weight percent between about, e.g., 1.0% to 10%, 10% to 25%, 15% to 35%, 0.5% to 5%, 0.001% to 1%, 0.01% to 1%, 0.1% to 1%, or 0.5% to 1%. In embodiments, the excipient is present at a weight percent between about, e.g., 0.001% to 1%, 0.01% to 1%, 1.0% to 5%, 10% to 15%, or 1% to 15%.

In embodiments, parenteral compositions may be administered as needed, e.g., once, twice, thrice or four or more times daily, or continuously depending on the subject's needs.

In embodiments, parenteral compositions of captodiamine or a pharmaceutically acceptable salt thereof are provided, wherein the pH of the composition is between about 4.0 to about 8.0. In embodiments, the pH of the compositions is between, e.g., about 5.0 to about 8.0, about 6.0 to about 8.0, about 6.5 to about 8.0. In embodiments, the pH of the compositions is between, e.g., about 6.5 to about 7.5, about 7.0 to about 7.8, about 7.2 to about 7.8, or about 7.3 to about 7.6. In embodiments, the pH of the aqueous solution is, e.g., about 6.8, about 7.0, about 7.2, about 7.4, about 7.6, about 7.7, about 7.8, about 8.0, about 8.2, about 8.4, or about 8.6.

As used herein, the term “pharmaceutically acceptable” refers to molecular entities and compositions that are “generally regarded as safe”, e.g., that are physiologically tolerable and do not typically produce an allergic or similar untoward reaction when administered to a human. In embodiments, this term refers to molecular entities and compositions approved by a regulatory agency of the federal or a state government, as the GRAS list under section 204(s) and 409 of the Federal Food, Drug and Cosmetic Act, that is subject to premarket review and approval by the FDA or similar lists, the U.S. Pharmacopeia or another generally recognized pharmacopeia for use in animals, and more particularly in humans.

As used herein, the term “pharmaceutically acceptable salts” includes acid addition salts, addition salts of free bases, wherein the compound is modified by making acid or base salts thereof. Examples of pharmaceutically acceptable salts include but are not limited to mineral or organic acid salts of basic residues such as amines, and alkali or organic salts of acidic residues such as carboxylic acids. Pharmaceutically acceptable salts include conventional non-toxic salts or quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, and oxalic salts. In embodiments, eplivanserin or pharmaceutically acceptable salts may include a hemifumarate salt. The pharmaceutically acceptable salts of captodiamine can be synthesized from the parent compound, which contains a basic or acidic moiety, by conventional chemical methods.

The terms “about” or “approximately” as used herein mean within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, a range up to 10%, a range up to 5%, and/or a range up to 1% of a given value. Alternatively, particularly with respect to biological systems or processes, the term can mean within an order of magnitude, e.g., within 5-fold, or within 2-fold, of a value. “About” and “approximately” are used interchangeably herein.

Patient in need thereof” includes individuals that have been diagnosed with ADHD. The methods may be provided to any individual including, e.g., wherein the patient is a neonate, infant, a pediatric patient (6 months to 12 years), an adolescent patient (age 12-18 years) or an adult (over 18 years). “Patient” and “subject” are used interchangeably herein. It should be understood that infants can receive a pediatric dose.

### EXAMPLE

The Example provided herein is included solely for augmenting the disclosure herein and should not be considered to be limiting in any respect.

### Example 1

### Prospective Assessment of Captodiamine in the Treatment of Adults with ADHD

This will be a randomized, double-blind, placebo-controlled, parallel-group, multicenter study in adult subjects with ADHD. Eligible subjects will be randomly assigned in a 1:1 ratio to one of two treatment groups: 100 mg captodiamine and placebo. The study will consist of three periods: a screening period of up to 2 weeks, a 6-week double-blind treatment period, and a 2-week safety follow-up period. The total duration of subject participation will be ˜10 weeks.

### Overview of Study Visits

### Screening Period:

Visit 1—Screening/Baseline Visit (up to 14 days prior to dosing)

### Treatment Period:

Visit 2—Day 0 (Randomization Visit) Visit 3—Day 7±2 days Visit 4—Day 14±2 days Visit 5—Day 28±2 days Visit 6—Day 42±2 days
Follow-up period:
Visit 7—Day 56±3 days

Eligible subjects will be randomly assigned to receive captodiamine 50 mg twice a day for a duration of 6 weeks. Eligible subjects will be randomly assigned to receive placebo twice daily for a duration of 6 weeks.

Primary outcome measures: Conners' Adult ADHD Rating Scales (CAARS™) [Time Frame: 6 weeks (from visit 1 baseline to visit 6)]. The primary efficacy endpoint is the difference in change (decrease) in CAARS (Total ADHD Symptoms Score) between the study groups. The CAARS assess the presence and severity of ADHD symptoms and behaviors in adults. Respondents are asked to report their own experiences by rating items pertaining to their behavior/problems using a 4-point Likert-style format ranging from 0 (‘Not at all’, ‘never’) to 3 (‘Very much’, ‘very frequently’). The scale measures ADHD symptoms using a 30-item questionnaire. Total score is the sum of all the items, min=30 Max=90.

Secondary outcome measures:
1. Test of Variables of Attention (TOVA) (Change in ADHD Score From Screening to Visit 6) [Time Frame: 6 weeks(visit 1 baseline to visit 6)]. The TOVA is a computerized test that provides information about an individual's sustained attention, speed and consistency of responding, and behavioral self-regulation and executive functioning. ADHD score is a comparison of the subject's response to the CPT test to those of an ADHD group, and is reported as a Z-score. An ADHD score of −1.80 and less fits the profile of the ADHD sample. A score of more than −1.80 (more positive) does not fit the ADHD profile. When comparing ADHD scores the higher the ADHD score the better the performance.
2. Adult ADHD Quality of Life (AAQoL)—Measuring Change in Total Score of AAQoL From Visit 1 to Visit 6 [Time Frame: 6 weeks (from visit 1 baseline to visit 6)]. The AAQoL scale provides a validated disease-specific measure of the impact of ADHD on quality of life. It is scored as an overall score (29 items) and four sub scale scores: life productivity (11 items), psychological health (6 items), life outlook (7 items) and relationships (5 items). Individual items are scored on a five-point Likert-like scale from ‘Not at all/Never’ (1) to ‘Extremely/Very Often’ (5).
3. Clinical Global Impression Scale (CGI-I)Score [Time Frame: 6 weeks from visit 1 baseline to visit 6]. The CGI is rated on a 7-point scale, with the severity of illness scale using a range of responses from 1 (normal) through to 7 (amongst the most severely ill patients). CGI-I scores range from 1 (‘very much improved’) through to 7 (‘very much worse’).

Those skilled in the art will recognize, or be able to ascertain using no more than routine experimentation, many equivalents to the embodiments described herein. Such equivalents are intended to be encompassed by the following claims.

What is claimed is:

1. A method of treating Attention-Deficit Hyperactivity Disorder (ADHD) comprising administering to a patient in need thereof a pharmaceutical composition comprising captodiamine or a pharmaceutically acceptable salt thereof.

2. The method of claim 1, wherein the composition provides reduction in hyperactivity, impulsiveness, inattentiveness, or a combination of symptoms in a patient diagnosed with ADHD.

3. The method of claim 1, wherein the captodiamine or a pharmaceutically acceptable salt thereof is administered in an amount of 0.01 mg to 1500 mg.

4. The method of claim 3, wherein the amount of captodiamine or a pharmaceutically acceptable salt thereof is from 10 mg to 250 mg.

5. The method of claim 3, wherein the amount of captodiamine or a pharmaceutically acceptable salt thereof is from 25 mg to 100 mg.

6. The method of claim 1, wherein the method provides improvement in one or more symptoms of the ADHD.

7. The method of claim 6, wherein the improvement is provided for more than 6 hours after administration.

8. The method of claim 6, wherein the method provides improvement in one or more symptoms of the ADHD the next day after administration of the composition.

9. The method of claim 1, wherein administering is accomplished via oral route, buccal route, sublingual route, rectal route, topical route, intranasal route, ophthalmic route, vaginal route or parenteral route.

10. The method of claim 1, wherein the total amount of captodiamine or a pharmaceutically acceptable salt thereof administered to the patient in a twenty-four hour period is between 1 mg and 500 mg.
